(19) United States
(12) Patent Application Publication
(10) Pub. No.: US 2024/0023999 A1
(43) Pub. Date: Jan. 25, 2024

(54) IMPLANTABLE DISTRACTION DEVICE AND METHOD OF INSTALLATION THEREOF

(71) Applicant: GLOBUS MEDICAL, INC., AUDUBON, PA (US)

(72) Inventor: Kurt Faulhaber, Cape Coral, FL (US)

(21) Appl. No.: 17/813,424

(22) Filed: Jul. 19, 2022

(51) Int. Cl. A61B 17/70

(52) U.S. Cl. CPC A61B 17/7068; A61B 2017/681

(57) ABSTRACT

The present disclosure provides an implantable distraction device capable of being installed between adjacent vertebrae, thereby facilitating distraction and pain relief. The distraction device may have two arms separated by a middle section. Each arm may be inserted to engage a spinous process. The implant, when inserted, provides distraction of the spinous process via a spring force that is exerted on each spinous process.

### FIELD

The present disclosure relates to the apparatus and method for allowing decompression of adjacent vertebral bodies to relieve pain, and more particularly relates to a pre-bent spinal implant capable of being inserted between adjacent vertebrae.

### BACKGROUND

A common procedure for handling pain associated with intervertebral discs that have become degenerated due to various factors such as trauma or aging is the use of intervertebral fusion devices for fusing one or more adjacent vertebral bodies. Generally, to fuse the adjacent vertebral bodies, the intervertebral disc is first partially or fully removed. An intervertebral fusion device is then typically inserted between neighboring vertebrae to maintain normal disc spacing and restore spinal stability, thereby facilitating an intervertebral fusion.

There are a number of known conventional fusion devices and methodologies in the art for accomplishing the intervertebral fusion. These include screw and rod arrangements, solid bone implants, and fusion devices which include a cage or other implant mechanism which, typically, is packed with bone and/or bone growth inducing substances. These devices are implanted between adjacent vertebral bodies in order to fuse the vertebral bodies together, alleviating the associated pain.

There are drawbacks associated with the known conventional fusion devices and methodologies and prior to a treatment plan involving spinal fusion, other treatment options may be preferable. These options may include more conservative approaches to alleviate the pain such as physical therapy, spinal manipulation, NSAIDs, pain medication, and/or epidural injections. It may be also preferable to determine with certainty, the level of the spine that is the cause of the pain.

As such, there exists a need for a diagnostic device that allows distraction to be applied to a single level of the spine insitu without fusion of adjacent vertebrae. The device may be a temporary implant to diagnose the cause of pain and identify the level of the spine causing the pain. After identification, conservative treatments without fusion may be used according to a treatment plan.

### SUMMARY OF THE DISCLOSURE

In an exemplary embodiment, the present disclosure provides an implant for use in a surgery directed to a spine of a patient. The implant includes a first arm having a first pocket. The first pocket may engage a first spinous process. The implant includes a second arm having a second pocket. The second pocket may engage a second spinous process. The implant includes a middle portion disposed between the first arm and the second arm. The implant is inserted into a patient, the first arm and the second arm may provide a spring force against the first spinous process and the second spinous process.

In another exemplary embodiment a surgical method to provide decompression of adjacent vertebral bodies. The method includes creating an incision in a patient to access the adjacent vertebral bodies and providing an implant. The implant includes a first arm having a first pocket, wherein the first pocket may engage a first spinous process. The implant includes a second arm having a second pocket, wherein the second pocket may engage a second spinous process. The implant further includes a middle portion disposed between the first arm and the second arm. The method further includes inserting the implant into a patient. The first pocket engages the first spinous process and the second pocket engages the second spinous process. A spring force is exerted against the first spinous process and the second spinous process.

### BRIEF DESCRIPTION OF THE DRAWINGS

The present invention will become more fully understood from the detailed description and the accompanying drawings, wherein:

FIG. 1A is a perspective view of an implant consistent with the principles of the present disclosure.

FIG. 1B is a perspective view of an implant consistent with the principles of the present disclosure.

FIG. 2A is a perspective view an incision for minimally invasive surgery for inserting an implant consistent with the principles of the present disclosure.

FIG. 2B is a perspective view of a spine subject to a minimally invasive surgery for inserting an implant consistent with the principles of the present disclosure.

FIGS. 3A, 3B, 3C, and 3D illustrate an implant consistent with the principles of the present disclosure implanted between adjacent vertebral bodies.

FIGS. 4A and 4B illustrate an implant consistent with the principles of the present disclosure implanted between adjacent vertebral bodies.

### DETAILED DESCRIPTION OF THE DISCLOSURE

The following description of the embodiment(s) is merely exemplary in nature and is in no way intended to limit the invention, its application, or uses.

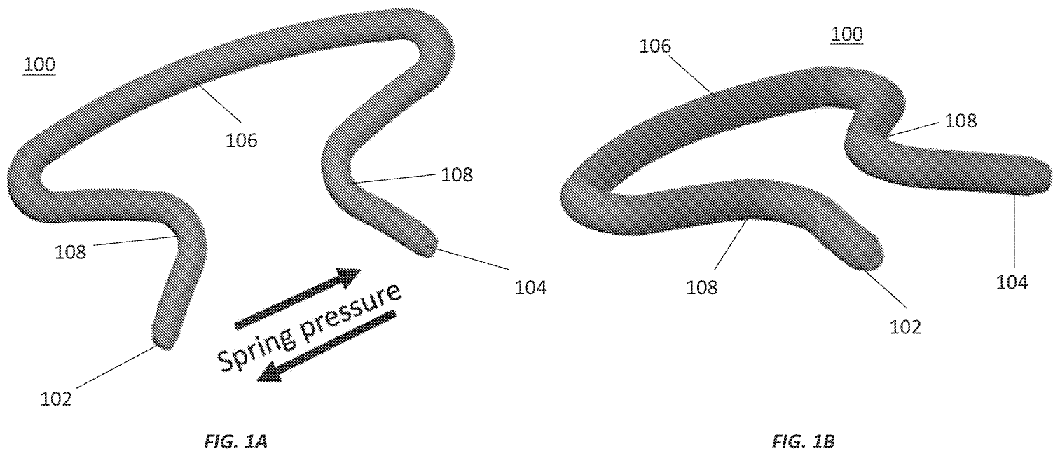

FIGS. 1A and 1B illustrate a distraction device or implant 100 consistent with the principles of the current disclosure. Implant 100 may be a pre-bent implant configured to be implanted between adjacent vertebral bodies, such as between adjacent spinous processes beneath the supraspinous ligament, splitting the interspinous ligament, and resting along the erector spinae muscle. Implant 100 may be fabricated from Nitinol. Implant 100 may be configured to have a spring pressure in order to provide the distraction of the adjacent vertebrae.

For example, implant 100 may have an arm 102 and an arm 104, wherein the arms 102 and 104 are separated from each other by a middle portion 106. Each of arms 102 and 104 may have a pocket 108 configured to engage a portion of a spinous process. As a force on arm 102 and arm 104 is made to move the arms closer to each other, implant 100 is configured to provide a spring pressure opposing that force. As arm 102 is disposed at one spinous process and arm 104 is disposed on an adjacent spinous process, the spring pressure acts to distract the adjacent spinous processes.

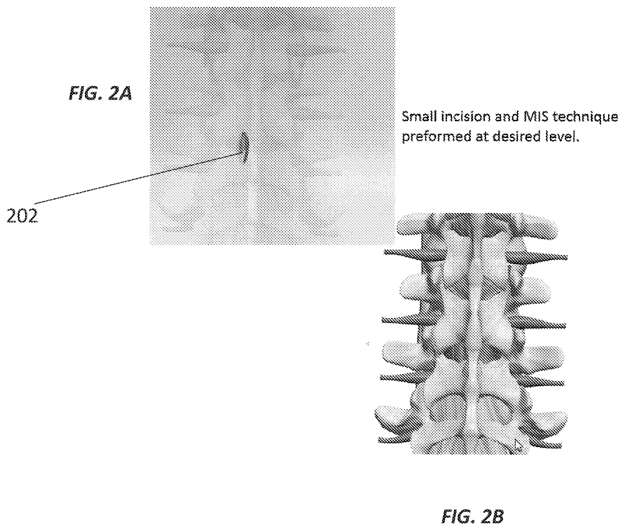

As part of a medical procedure to insert implant 100 into a patient, a desired level of the spine is determined by a surgeon. After the desired level of the spine for the distraction, for example between adjacent lumbar spine vertebrae, a small incision 202 may be conducted on the patient as part of a minimally invasive surgery (MIS), as shown in FIG. 2A. FIG. 2B illustrates the adjacent vertebral bodies at or near the determined level of the spine for distraction.

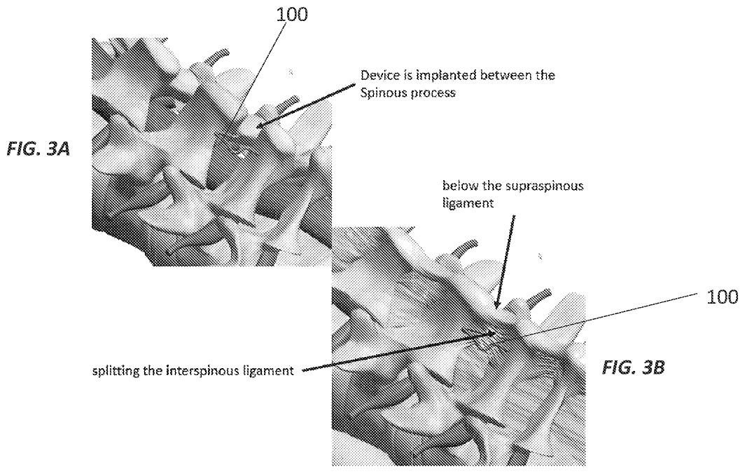

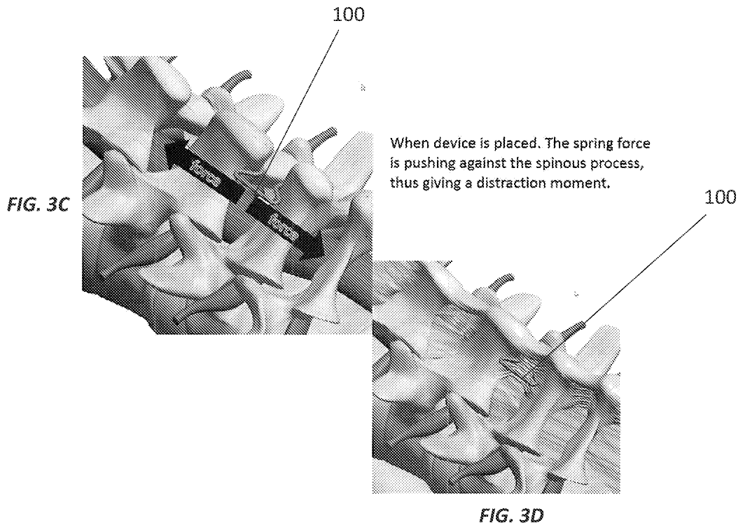

As the desired level of the spine, implant 100 is inserted between the adjacent spinous processes, as shown in FIG. 3A. Implant 100 is disposed below the supraspinous ligament and is shown splitting the interspinous ligament in FIG. 3B. As shown in FIGS. 3C and 3D, when implant 100 is placed in the desired location, a spring force is exerted on the adjacent spinous processes in opposite directions thereby providing distraction of the spinous processes.

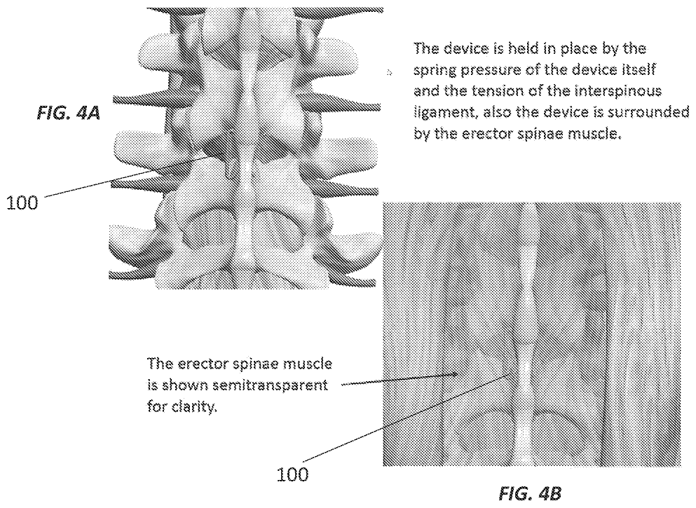

As shown in FIGS. 4A and 4B, implant 100 is configured to be held in place by the spring pressure of the inserted implant 100 as well as tension of the interspinous ligament. Also contributing to holding implant 100 in place is the erector spinae muscle of the patient surrounding inserted implant 100. As mentioned above, implant 100 may be temporary inserted in order for the surgeon to determine the proper level of the spine that is causing pain and determining conservative approaches for treatment such as physical therapy, spinal manipulation, NSAIDs, pain medication, and/or epidural injections.

Benefits of implant 100 include that it may be used a diagnostic device to determine where pain is originating. The use of implant 100 may be preferable to more permanent solutions such as fusion of the adjacent vertebrae, which may also be more invasive than the principles disclosed herein. Use of implant 100 may help alleviate pain and facilitate long term healing with more conservative treatments than fusion surgery.

The disclosure being thus described, it will be obvious that the same may be varied in many ways. Such variations are not to be regarded as a departure from the spirit and scope of the disclosure, and all such modifications as would be obvious to one skilled in the art are intended to be included within the scope of the following claims.

1. An implant for use in a surgery directed to a spine of a patient, comprising:
a first arm having a first pocket, wherein the first pocket is configured to engage a first spinous process;
a second arm having a second pocket, wherein the second pocket is configured to engage a second spinous process; and
a middle portion disposed between the first arm and the second arm,
wherein when the implant is inserted into a patient, the first arm and the second arm are collectively configured to provide a spring force against the first spinous process and the second spinous process,
wherein the implant does not extend beyond a middle portion of the first or second spinous process,
wherein the implant is positioned asymmetrical to a central longitudinal axis of the spinous process.

2. The implant of claim 1, wherein the implant is pre-bent.

3. The implant of claim 1, wherein the implant is fabricated from Nitinol.

4. The implant of claim 1, wherein the first arm and the second arm are configured to be disposed between adjacent lumbar vertebral bodies.

5. The implant of claim 1, wherein the implant is configured to be disposed beneath a supraspinous ligament.

6. The implant of claim 5, wherein the implant is configured to split an interspinous ligament.

7. The implant of claim 6, wherein the implant is configured to rest along an erector spinae muscle of the patient.

8. The implant of claim 1, wherein the implant is configured to be temporarily inserted into the patient to determine a level of the spine causing pain.

9. The implant of claim 1, wherein the implant is configured to be inserted into the patient via a minimally invasive surgical (MIS) procedure.

10. A surgical method to provide decompression of adjacent vertebral bodies, said method comprising:
creating an incision in a patient to access the adjacent vertebral bodies;
providing an implant, the implant including:
a first arm having a first pocket, wherein the first pocket is configured to engage a first spinous process;
a second arm having a second pocket, wherein the second pocket is configured to engage a second spinous process; and
a middle portion disposed between the first arm and the second arm,
inserting the implant into a patient, wherein the first pocket engages the first spinous process and the second pocket engages the second spinous process, and wherein a spring force is exerted against the first spinous process and the second spinous process
wherein the implant does not extend beyond a middle portion of the first or second spinous process,
wherein the implant is positioned asymmetrical to a central longitudinal axis of the spinous process.

11. The method implant of claim 10, wherein the implant is pre-bent.

12. The method of claim 10, wherein the implant is fabricated from Nitinol.

13. The method of claim 10, wherein the first arm and the second arm are disposed between adjacent lumbar vertebral bodies.

14. The method of claim 10, wherein the implant is disposed beneath a supraspinous ligament.

15. The method of claim 10, wherein the implant is configured to split an interspinous ligament.

16. The method of claim 10, wherein the implant is configured to rest along an erector spinae muscle of the patient.

17. The method of claim 10, wherein the implant is temporarily inserted into the patient to determine a level of the spine causing pain.

18. The method of claim 10, wherein the implant is inserted into the patient via a minimally invasive surgical (MIS) procedure.
